(19) United States
(12) Patent Application Publication
(10) Pub. No.: US 2016/0096011 A1
(43) Pub. Date: Apr. 7, 2016

(54) CHEMOTHERAPY MEDICATION HOLDER

(71) Applicant: Ivan Stovall, Richmond, VA (US)

(72) Inventor: Ivan Stovall, Richmond, VA (US)

(21) Appl. No.: 14/508,139

(22) Filed: Oct. 7, 2014

(51) Int. Cl. A61M 39/08; A61M 5/142

(52) U.S. Cl. CPC A61M 39/08; A61M 5/14244; A61M 2209/088

(57) ABSTRACT

A chemotherapy medication holder is worn discretely to secure a patient's chemotherapy medications close to their body without hindering their range of motion. The chemotherapy medication holder comprises an elongated strap, where the elongated strap fastens around a user; a fastener at each end of the elongated strap, where the fasteners enable size adjustments of the elongated strap for a comfortable fit; a pocket coupled to the elongated strap, where the pocket receives a container of chemotherapy medication; and a tubing pocket against the pocket, where the tubing pocket receives one or more tubes from the container of chemotherapy medication.

### BACKGROUND OF THE INVENTION

1. Field of the Invention

The present invention relates to a holder to secure a medication pump and the associated tubes close to a user's body thereby enabling free movement without obstruction.

2. Description of Related Art

Chemotherapy is a method of cancer treatment that invades the body with drugs and medications that kill rapidly dividing cells, like cancer cells. Because other types of cells are also rapidly dividing, like those in bone marrow, the digestive rack and hair follicles, side effects like hair loss, and decreased production of blood cells are commonly associated with patients undergoing cancer treatment. Typically, the patient receives their treatments through intravenous administration which allows the medication to rapidly enter the bloodstream. The patient must sit and wait while they are tethered to the machines until their treatment is finished. They must find stationary ways occupy themselves for hours while they receive their life saving treatments.

Recently, some patients are able to wear portable chemotherapy pumps. Often these pumps are used to administer the drugs continuously over an extended period of time. This permits the patient to go about their daily routine without having to stay in a hospital for their entire treatment. While the pumps are convenient and portable, they are often cumbersome and bulky, limiting the person's movement while wearing. Additionally, the tubes associated with the pumps often tangle around the person's limbs, clothing and objects. This may be damaging to both the tube and to the items caught.

Therefore it would be beneficial in the art to provide a device that allows a chemotherapy patient to discretely and comfortably wear their medication pump. It would also be desirable in the art to provide a device that prevents the medication tubes from becoming tangled or pinched while receiving portable cancer treatments.

### SUMMARY OF THE INVENTION

In view of the foregoing disadvantages inherent in the prior art, the general purpose of the present invention is to provide a chemotherapy medication holder is worn discretely to secure a patient's chemotherapy medications close to their body without hindering their range of motion, configured to include all of the advantages of the prior art, and to overcome the drawbacks inherent therein.

Accordingly, an object of the present invention is to provide a chemotherapy medication holder that straps around a patient's waist or torso to discretely wear their portable pump within a pocket attached to the holder.

Another object of the present invention is to provide a chemotherapy medication holder that includes an additional pocket to contain the tubes that administer the medication, therefore preventing the tubes from being seen and tangling.

To achieve the above objects, in an aspect of the present invention, a chemotherapy medication holder is described comprising an elongated strap, where the elongated strap fastens around a user; a fastener at each end of the elongated strap, where the fasteners enable size adjustments of the elongated strap for a comfortable fit; a pocket coupled to the elongated strap, where the pocket receives a container of chemotherapy medication; and a tubing pocket against the pocket, where the tubing pocket receives one or more tubes from the container of chemotherapy medication.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWING

The advantages and features of the present invention will become better understood with reference to the following detailed description and claims taken in conjunction with the accompanying drawing in which:

FIG. 1 depicts a perspective view of a chemotherapy medication holder in accordance with an exemplary embodiment of the present invention.

### DETAILED DESCRIPTION OF THE DRAWING

The present invention relates to a holder to secure a medication pump and the associated tubes close to a user's body thereby enabling free movement without obstruction. The present invention provides a chemotherapy medication holder to facilitate the process of administering chemotherapy treatments. The chemotherapy medication holder includes an elongated belt with an attached pocket. The belt fastens around the patient's waist, positioning the pockets at a convenient level to comfortably hold the pump and tubes associated with chemotherapy treatments. The chemotherapy medication holder may be adjusted as needed to discretely and effectively support and conceal the tubes and accessories.

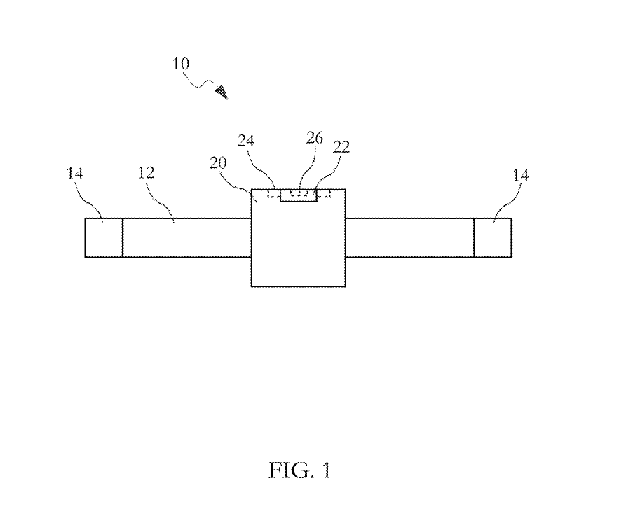

Turning now descriptively to the drawing, referring to FIG. 1, a perspective view of a chemotherapy medication holder 10 is shown in accordance with an exemplary embodiment of the present invention. The chemotherapy medication holder 10 includes an elongated strap 12 or belt. The elongated strap 12 may wrap around a user's waist or torso, wherever preferred to be comfortably worn by a chemotherapy patient. At each end of the elongated strap 12 is a fastener 14. The fasteners 14 may be Velcro®, also referred to as a hook and loop fastener, a buckle, snaps, buttons or the like to enable the user to repeatedly adjust the elongated strap 12 as needed for comfort and stability. The elongated strap 12 may be made from elastic, neoprene, nylon, rubber or the like, and be decorated with a variety of prints and colors for an aesthetic appeal to the user. The elongated strap 12 may range in length from 30-70 inches in length to accommodate patients of all sizes.

Attached along the elongated strap 12 is a first pocket 20. The first pocket 20 may receive a pump or bottle dispenser that delivers chemotherapy treatment medications. A middle portion of the first pocket 20 may have a tubing pocket 22 to receive the tubes extending from the medication pump. The tubing pocket 22 may be positioned within the first pocket 20 to keep the tubes from getting tangled around the dispenser. Alternatively, the tubing pocket 22 may be positioned next to the first pocket 20 but on the exterior portion. This ensures that the tubing pocket 22 interfere with the dispenser. The first pocket 20 may be 4-8 inches in length and 4-8 inches in height, preferably 5.75 inches by 6.6 inches. The tubing pocket 22 may measure 2 inches square. Along the top portion of the first pocket 20 may be a closure 24 to prevent the pump or bottle from exiting the first pocket 20. Similarly, the tubing pocket 22 may have a closure 26 to keep the tubing securely fastened to the holder 10. The closures 24, 26 may be hook and loop fasteners for easy and repeated opening and closing. Finally, each pocket 20, 22 may be lined with a waterproof material to prevent any medications or other liquids from getting onto the user's exterior clothing. With the chemotherapy medication holder 10 the patient is afforded free range of movements while inconspicuously receiving treatments. The adjustable nature of the elongated strap 12 allows the user to adjust the length as needed for optimal comfort.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A chemotherapy medication holder comprising:
a. an elongated strap, where the elongated strap fastens around a user;
b. a fastener at each end of the elongated strap, where the fasteners enable size adjustments of the elongated strap for a comfortable fit;
c. a first pocket coupled to the elongated strap, where the pocket receives a container of chemotherapy medication; and
d. a tubing pocket against the pocket, where the tubing pocket receives one or more tubes from the container of chemotherapy medication.

2. The chemotherapy medication holder according to claim 1, where the fasteners are one of at least a hook and loop fastener, a buckle, snaps or buttons.

3. The chemotherapy medication holder according to claim 1, where the first pocket and the tubing pocket each include a closure.

4. The chemotherapy medication holder according to claim 3, where the closure is a hook and loop fastener.

5. The chemotherapy medication holder according to claim 1, where the first pocket and the tubing pocket are lined with a waterproof material.

6. The chemotherapy medication holder according to claim 1, where the tubing pocket is positioned within the first pocket.

7. The chemotherapy medication holder according to claim 1, where the first pocket measures 5.75 inches in length by 6.5 inches in height.
